(19) United States
(12) Patent Application Publication
(10) Pub. No.: US 2020/0181044 A1
(43) Pub. Date: Jun. 11, 2020

(54) Process and facility for producing propylene by combining propane dehydrogenation and a steam cracking method with propane recirculation into the steam cracking method

(71) Applicant: LINDE AKTIENGESELLSCHAFT, München (DE)

(72) Inventors: Torben HÖFEL, München (DE); Christine TÖGEL, München (DE); Mathieu ZELLHUBER, Planegg (DE); Heinrich LAIB, Ludwigshafen (DE); Stefan KOTREL, Ludwigshafen (DE); Martin DIETERLE, Ludwigshafen (DE); Florina Corina PATCAS, Ludwigshafen (DE); Sonja GIESA, Ludwigshafen (DE)

(21) Appl. No.: 16/630,361

(22) PCT Filed: Jul. 12, 2018
(86) PCT No.: PCT/EP2018/068967

§ 371 Date: Jan. 10, 2020

(30) Foreign Application Priority Data
Jul. 12, 2017 (EP) 17180984.1

(51) Int. Cl. C07C 5/333; C07C 7/00; C07C 7/04; C07C 7/09; B01J 19/24; C10G 9/36

(52) U.S. Cl. CPC C07C 5/333; C07C 7/005; C07C 7/04; C07C 7/09; B01J 19/245; C10G 2300/4056; B01J 2219/0004; C10G 2300/104; C10G 2300/1081; C10G 2400/20; C10G 9/36

(57) ABSTRACT

The invention relates to a process (10) for the production of propylene which comprises carrying out a process (1) for propane dehydrogenation to obtain a first component mixture (A), carrying out a further propylene production method (2) to obtain a second component mixture (B), and forming a separation product (P2) containing predominantly propane using one or more propane separation steps (S1), wherein at least part of the first component mixture (A) is supplied to the propane separation step or steps (S1). It is envisaged that the separation product (P2), which mainly contains propane, will at least partly be returned to the further propylene production method (2). A corresponding plant and a process for converting a steam cracking plant are also the subject of the invention.

Process and facility for producing propylene by combining propane dehydrogenation and a steam cracking method with propane recirculation into the steam cracking method

The present invention relates to a process and a plant for the production of propylene and to a process for retrofitting a steam cracking plant according to the preambles of the independent claims.

### PRIOR ART

Propylene (propene) is traditionally produced mainly by steam cracking of hydrocarbon feedstocks and other conversion methods in refinery processes. In these cases propylene is a minor by-product. Due to the increasing demand for propylene, especially polypropylene, propane dehydrogenation is also used.

Propane dehydrogenation is a well-known method in the petrochemical industry and is described in the article “Propene” in Ullmann's Encyclopedia of Industrial Chemistry, Online Edition 16 Sep. 2013, DOI: 10.1002/14356007.a22_211.pub3, in particular in Section 3.3.1, “Propane dehydrogenation”.

Propane dehydrogenation is an endothermic equilibrium reaction generally carried out on noble or heavy metal catalysts, such as platinum or chromium. The dehydrogenation reaction is highly selective. For commercially available processes, total yields of approx. 90% are cited. Notwithstanding this high selectivity, smaller quantities of hydrocarbons with one, two, four and more than four carbon atoms are typically produced as by-products in addition to the hydrogen which is cleaved off. These by-products must be separated from the target product propylene.

Steam cracking methods and refinery processes in which propylene is formed are also described in literature, for example in the article “Ethylene” in Ullmann's Encyclopedia of Industrial Chemistry, online publication 15 Apr. 2009, DOI: 10.1002/14356007.a10_045.pub3, and in the article “Oil Refining” in Ullmann's Encyclopedia of Industrial Chemistry, online publication 15 Jan. 2007, DOI: 10.1002/14356007.a18_051.pub2.

In principle, the purification of a component mixture produced during propane dehydrogenation can at least partly be carried out together with the purification of a component mixture containing propylene from another method in which propylene is formed, e.g. a steam cracking method or a refinery process.

The combination of the purification of a component mixture from a propane dehydrogenation with the purification of a component mixture from a steam cracking method is known, for example, from U.S. Pat. No. 4,458,096 A or, specifically related to a cracking of hydrocarbons with two carbon atoms, from WO 2015/128039 A1, and a corresponding combination with the purification of a component mixture from a fluid catalytic cracking method for example from U.S. Pat. No. 8,563,793 A or US 2010/331589 A1. However, these publications do not contain more detailed information regarding a corresponding combination. It should also be noted at this point that a fluid catalytic cracking method provides product mixtures with a fundamentally different composition than a steam cracking method, so that a combined separation must be designed differently here.

The aforementioned WO 2015/128039 A1 describes that a component mixture with predominantly or exclusively two carbon atoms, which is formed in a common separation, to which a component mixture formed using the propane dehydrogenation method and a component mixture formed using the steam cracking method are subjected, is returned to the steam cracking method. On the other hand, a component mixture with three or more carbon atoms formed in the joint separation is returned to the propane dehydrogenation method because it contains considerable amounts of propane. Other publications also reveal a return of propane or mixtures of components containing propane to a propane dehydrogenation method.

The present invention has the task of improving and making more efficient processes for the production of propylene, in which a component mixture is purified from a propane dehydrogenation method and in which certain components or fractions are recycled, if an additional steam cracking method is available.

### DISCLOSURE OF THE INVENTION

Against this background, the present invention proposes a process and plant for the production of propylene and a process for retrofitting a plant to perform a steam cracking method with the respective characteristics of the independent claims. Preferred embodiments are the subject of the dependent claims as well as the following description.

The present invention recognizes that paradoxically, recycling a separation product predominantly containing propane not to the propane dehydrogenation method but to the other propane production method is advantageous when the separation product predominantly containing propane is formed in a separation to which at least a portion of a first component mixture provided using a propane dehydrogenation method and optionally also at least a portion of a second component mixture provided using a further propane production method are subjected. This is particularly the case where the other propane production method is a steam cracking method.

This recycling is counterintuitive, since the separation product, which mainly contains propane, is supposed to be a particularly valuable feed for the propane dehydrogenation method, since propane is to be converted into propylene in the propane dehydrogenation method and a propane-rich feed is therefore known to be used. Therefore, it would be very obvious, if a recycle is to take place, to return a propane-containing component mixture to the propane dehydrogenation method, as proposed in WO 2015/128039 A1 with regard to a component mixture containing propane and heavier hydrocarbons. The invention at hand has recognized just the opposite as advantageous.

Propane-containing feeds for propane dehydrogenation are typically present with a high propane concentration (e.g. at least 94 percent by volume propane and in each case at most 4 percent by volume butane, 3 percent by volume ethane, and 0.1 percent by volume olefins). Compared to this, a separation product from a separation which mainly contains propane, in particular if to this joint separation is also subjected a component mixture which is provided using a steam cracking method, may be of inferior quality. This applies at least if the separation does not involve a disproportionate effort in terms of the purity of the products obtained. A corresponding separation product predominantly containing propane therefore contains olefins, in particular mono- and polyunsaturated olefins with three carbon atoms and mono- and polyunsaturated olefins with four carbon atoms.

Depending on the design of the propane dehydrogenation method, the reactor or its catalyst used may have extremely small tolerances with respect to components such as methyl acetylene, propadiene and butadiene, which are regularly formed in corresponding other propylene production methods, in particular in steam cracking methods. A recycle to the propane dehydrogenation method would therefore require at least extensive separation of the above components in order to comply with the tolerances of an appropriate reactor. However, this would entail a disproportionately high level of technical and procedural effort.

A process according to a particularly preferred embodiment of the invention comprises a combined purification or separation of component mixtures from the propane dehydrogenation method and the steam cracking method. However, the process according to the invention can basically also be carried out without a combined purification or separation. For a combined purification of components from different methods it is particularly advantageous if the respective component mixtures contain identical or similar components, i.e. the component mixtures do not “contaminate” each other with certain components not contained in the respective other component mixture (e.g. with hydrogen, carbon dioxide or oxygenates).

A combination of purification is particularly advantageous if the respective component mixtures have a similar concentration range, so that a synergetic separation process can be expected. However, this is normally not the case in practice. Furthermore, a combination of purification is advantageous if one of the processes supplies significantly smaller quantities of a corresponding component mixture or if a corresponding plant is smaller and therefore separate purification is not worthwhile. This may be the case in particular if the other propylene production method, e.g. a steam cracking method, is already implemented in the form of a plant and a propane dehydrogenation method with significantly lower capacity is retrofitted to increase the propylene product capacity. This could be particularly advantageous if some plant components for the steam cracking method are no longer running at full capacity due to a later change in use and these capacities can be used by the propane dehydrogenation method.

If the steam cracking method is already designed for the use of a propane-containing feed, this can also be continued to be operated after retrofitting with propane. However, after retrofitting, the steam cracking method receives at least part of the separation product, which mainly contains propane, which does not have a major effect on the process. The propane dehydrogenation method obtains the higher quality propane feedstock from another source (and possibly also at least part of the separation product predominantly containing propane). Accordingly, the (common) separator does not have to be designed to meet the requirements of the propane dehydrogenation method with respect to the separation product predominantly containing propane, i.e. in particular no steps for (separate) hydrogenation and no steps for the separation of hydrocarbons with four or more carbon atoms are required in order to comply with the tolerances of the propane dehydrogenation method.

One possibility for advantageously designing the preferred embodiment of the present invention, in which a combined purification or separation is used, is therefore to pretreat a first component mixture, which is obtained using the propane dehydrogenation method, in such a way that it is present in a state depleted of (at least) hydrogen and in particular at an increased pressure. The first component mixture is subjected to one or more pre-separation steps, which are subsequently referred to as the “first” pre-separation steps. The component mixture pre-treated in this way, which is subsequently referred to as the “third” component mixture, contains mainly hydrocarbons with three carbon atoms due to its pre-treatment. It may also contain smaller quantities of methane, residual hydrogen and hydrocarbons containing two carbon atoms and hydrocarbons containing four carbon atoms and, where appropriate, more than four carbon atoms.

In the design of the preferred embodiment of the present invention described above, it is also envisaged to pretreat a second component mixture, which is obtained using the steam cracking method, in such a way that it is present in a state depleted (at least) of hydrogen and methane and, in particular, also at an increased pressure. Here, the second component mixture is subjected to one or more pre-separation steps, which are subsequently also referred to as “second pre-separation steps”. The component mixture pretreated in this way, which is hereinafter referred to as the “fourth” component mixture if specifically referred to here, advantageously contains, due to its pre-treatment, predominantly hydrocarbons similar to those contained in the third component mixture and in a comparable concentration range, as well as comparable quantities of residual hydrogen and residual methane, unless completely separated.

Since larger quantities of hydrocarbons with two carbon atoms, in particular ethane and ethylene, can generally be formed in a propylene production process used alternatively or in addition to a process for propane dehydrogenation, in particular a steam cracking method, in particular the use of lighter steam cracking inserts, a depletion of hydrocarbons with two carbon atoms can also take place in the course of the depletion of hydrogen and methane in the course of the second preliminary separation step or steps. In other words, a Demethanizer First process or a Deethanizer First process can be used in the course of the second pre-separation step or steps. The use of a Depropanizer-First process is also possible in principle. Further details are explained below. However, even in the course of the first preliminary separation step or steps, a depletion of hydrocarbons with two carbon atoms can take place if this is appropriate. Further aspects of these embodiments are explained below.

The composition and pressure of the third and fourth component mixtures, which have been at least partially adjusted to each other by the first pre-separation step or steps and by the second pre-separation step or steps, can be combined in a particularly advantageous manner and subsequently subjected to subsequent common separation steps. This makes it possible to design corresponding plant components for both processes together and thus to build a corresponding plant with lower investment costs and/or to operate it with lower operating costs.

Is there talk here of a component mixture compared to another component mixture, here especially the third compared to the first and the fourth compared to the second, at one or more components, here especially at hydrogen or hydrogen and methane “depleted” is understood to mean that the depleted component mixture contains at most 0.5 times, 0.2 times, 0.1 times, 0.01 times or 0.001 times the content of the one or more components relative to the nondepleted component mixture and on a molar, mass or volume basis. Also a complete distance, i.e. a “depletion to zero” is understood here as a depletion. In the following, the term “predominantly” refers to a content of at least 60%, 80%, 90%, 95% or 99% on a molar, mass or volume basis.

Altogether, the present invention proposes a process for the production of propylene which involves carrying out a propane dehydrogenation method to obtain a first component mixture which contains at least hydrogen, ethane, ethylene, propane and propylene, and carrying out a further propylene production method, in particular a steam cracking method, to obtain a second component mixture which contains at least hydrogen, methane, ethane, ethylene, propane and propylene. For the details of the respective processes and the product compositions typically formed, in particular also with regard to compounds contained in addition to the mentioned components, please refer to the technical literature cited several times. The propane dehydrogenation method is advantageously supplied with inserts containing propane and the steam cracking method with inserts rich in hydrocarbons. The latter are, for example, naphtha, but may also be lighter or heavier applications, i.e. those which contain hydrocarbons with a lighter and/or heavier boiling point than are typically present in naphtha.

As is customary in combined processes, a separation product predominantly containing propane is formed using at least part of the propane of the first component mixture and one or more propane separation steps. According to the preferred form of the present invention described above, the separation product predominantly containing propane can also be formed by using at least part of the propane of the second component mixture, i.e. by a common purification or separation. The propane separation step or steps may include in particular the use of a C3 splitter from which a separation product predominantly or exclusively containing propylene can be removed at the head and the separation product predominantly containing propane can be removed at the bottom. Such a C3 splitter is typically preceded by further separation steps, as explained below.

In particular, the feed of a corresponding C3 splitter can typically be taken from a so-called depropanizer or a corresponding depropanizer column, as is also generally known from the cited technical literature. A depropanizer is a rectification column from which a gaseous fraction containing predominantly or exclusively hydrocarbons with three carbon atoms can be withdrawn at the top and a liquid fraction containing predominantly or exclusively hydrocarbons with four and optionally more carbon atoms can be withdrawn at the bottom. From other apparatuses assigned to a corresponding rectification column which are part of a corresponding depropanizer, such as absorbers, such fractions can also be removed instead of the rectification column itself or in addition to it. The fraction taken from the head of the depropanizer or corresponding apparatus can be fed to the C3 splitter. A corresponding depropanizer may be arranged at different locations in a separation sequence for processing a component mixture, in particular a component mixture obtained by a steam cracking method or another propane production process. In particular, a corresponding depropanizer can be arranged downstream of a deethanizer or a corresponding deethanizer column within the scope of the present invention and set up for the separation processing of a bottom product of the deethanizer. As explained below, a deethanizer can be used in the course of the present invention in the course of the second pre-separation step or steps or already in the course of the propane separation step or steps, depending on whether a deethanizer-first process or a demethanizer-first process is used. The same applies to a depropanizer, which can also be at the first position of a corresponding separation sequence.

At least part of the first component mixture and, in accordance with the design with common purification or separation, at least part of the second component mixture is always added to the propane separation step or steps used in the context of the present invention. By “at least part” it shall also be understood that a fraction formed, for example, in the pre-separation step or steps described above, is added to the propane separation step or steps. In other words, a “part” does not have to be a partial amount with identical composition, but a “part” may also be a fraction with a different composition, in particular another mixture of components formed in a pre-separation step (first or second) already mentioned. In other words, at least a part (in terms of a partial amount) of the first component mixture and optionally at least a part (in terms of a partial amount) of the second component mixture can be supplied to the propane separation step or steps used in the present invention, but it is also possible to supply to the propane separation step or steps one or more component mixtures (namely in particular the already mentioned “third” and “fourth” component mixtures) formed using at least a part of the first component mixture and at least a part of the second component mixture.

The advantages of the essential aspect of the present invention, according to which the separation product, which mainly contains propane, is at least partly returned to the further propylene production method, have already been explained before. These consist, as mentioned above, in particular in the fact that in this way it can be avoided that incompatible components, in particular mono- and polyunsaturated hydrocarbons with three or more carbon atoms, are added to the propane dehydrogenation method. As also mentioned, in this way it is not necessary to subject the separation product, which mainly contains propane, to an elaborate cleaning process, since in the further propylene production method, in particular a steam cracking method, at least residual contents of corresponding components can be tolerated and, if necessary, even converted. In this way, the process proposed in accordance with the invention can be carried out more simply and cost-effectively, in contrast to other processes which feed the processes for propane dehydrogenation with corresponding recycled streams. For the propane dehydrogenation method, fresh feeds with corresponding specifications can always be used within the scope of the present invention.

If it is stated here that the separation product predominantly containing propane is “at least partly” returned to the further propylene production method, this also includes a constellation according to which part of the separation product predominantly containing propane is returned to the further propylene production method and another part of the separation product predominantly containing propane is returned to the propane dehydrogenation method. In this way, in the propane dehydrogenation method, the content of interfering components can be reduced in such a way that they no longer interfere with the process, for example by means of a corresponding “dilution” with fresh feed. In other words, the separation product, which mainly contains propane, can be admixed here to the extent that it does not interfere with the propane dehydrogenation method. The remainder of the separation product, which mainly contains propane, can, however, be returned to the further propylene production method. In accordance with a particularly preferred design of the present invention, however, the separation product predominantly containing propane is not returned to the propane dehydrogenation method, so that the propane dehydrogenation method in this design of the process conforming to the invention is supplied only with propane which does not originate from either the first or the second component mixture.

As mentioned above, the present invention does not require time-consuming cleaning due to the return of the separation product, which mainly contains propane. In other words, a separation product containing only predominantly propane may be used, which may contain from 0.1 to 25% by volume of monounsaturated and polyunsaturated hydrocarbons, in particular monounsaturated and polyunsaturated hydrocarbons containing three and four carbon atoms, in particular propadiene and butadiene. Such a separation product can be tolerated within higher limits in another propylene production method, in particular a steam cracking method, than in the propane dehydrogenation method.

As already mentioned, other products are typically created within the framework of a corresponding process. Thus, a process according to a particularly preferred embodiment of the present invention also includes the formation of a separation product containing predominantly or exclusively propylene using at least part of the problem of the first and second component mixture and using the propane separation step or steps. As already explained, a C3 splitter can be used in this context.

The present invention further comprises, in the embodiment just described, the formation of a separation product predominantly or exclusively containing ethylene using at least part of the ethylene of the first and second component mixtures and using one or more further separation steps, and the formation of a separation product predominantly or exclusively containing ethane using at least part of the ethane of the first and second component mixtures and using the further separation step or steps. The further separation step or steps typically include the use of a so-called C2 splitter, from which the separation product predominantly or exclusively containing ethylene can be taken at the top and the separation product predominantly or exclusively containing ethane can be taken at the bottom. A corresponding C2 splitter can in particular be fed with a fraction containing predominantly or exclusively ethane and ethylene which can be withdrawn from the head of a deethanizer, i.e. a corresponding rectification column or an apparatus associated therewith, in a demethanizer first process and from the bottom of a demethanizer in a deethanizer first process.

As mentioned above, the present invention comprises pre-separation steps according to particularly preferred embodiments, wherein it is provided that at least a part of the first component mixture is subjected to one or more first pre-separation steps to obtain a third component mixture, which comprises a pressure increase and an at least partial removal of hydrogen, and that at least a part of the second component mixture is subjected to one or more second pre-separation steps to obtain a fourth component mixture, which comprises a pressure increase, an at least partial removal of hydrogen and an at least partial removal of methane. It should already be noted at this point that, as mentioned above, at least partial removal of hydrocarbons with two carbon atoms can also be carried out in the context of the second pre-separation step or steps. In the latter case, the present invention is used in connection with a deethanizer-first method, otherwise in connection with a demethanizer-first method. As mentioned above, this invention can also be used in conjunction with a Depropanizer-First process.

In addition, the present invention provides, in accordance with the embodiments described above, that at least part of the third component mixture, together with at least part of the fourth component mixture, is subjected to the propane separation step or steps in an alternative. According to a further alternative, it is envisaged that at least part of the third component mixture together with the second component mixture is subjected to the second pre-separation step or steps to form the fourth component mixture and that the fourth component mixture is subjected to the propane separation step or steps. As already mentioned, the third and fourth component mixtures can be “parts” of the first and second component mixtures, respectively, in the sense explained above. The present invention, with its respective embodiments explained below, provides different possibilities for combining the third component mixture with the fourth component mixture or the respective proportions used. In all cases, the main advantage of the present invention is that a particularly simple and efficient joint separation is possible due to a comparable composition of the third and fourth component mixtures.

As already mentioned, this invention can be used in conjunction with a deethanizer-first process. In this case, when hydrogen and methane are removed in the second pre-separation step or steps, ethane and ethylene are also at least predominantly removed, so that these do not predominantly transition from the second to the fourth component mixture or corresponding proportions. It should be noted that in a corresponding deethanizer-first process, the fourth component mixture can also be a liquid that is present on a separating tray near the bottom of a corresponding rectification column. A “separating tray near the bottom” is a separating tray arranged in the lower half, especially in the lower third, in the lower quarter or in the lower fifth of the rectification column.

In this context, a “rectification column” is a separation unit which is set up to at least partially separate one or more gaseous or liquid component mixtures, or in the form of a two-phase mixture with liquid and gaseous components, possibly also in the supercritical state, by rectification, i.e. to produce pure substances or at least component mixtures with a different composition from the component mixture(s). Rectification is known to involve repeated evaporation and condensation processes, especially on or using suitable internals such as separating trays or ordered or disordered packings. A rectification column for use within the scope of this invention has a bottom evaporator. This is a device with a heat exchanger that is heated and is designed to heat a liquid fraction, also known as a bottom liquid, that accumulates in the bottom of the rectification column. By means of a bottom evaporator, part of the bottom liquid is continuously evaporated and fed back into the rectification column in gaseous form. A rectification column for use in the context of the present invention also contains a head condenser which condenses gas rising in the rectification column and returns it to the rectification column in a condensed state.

For the design and specific configuration of rectification columns and other separation equipment, reference is made to relevant textbooks (see, for example, K. Sattler, “Thermische Trennverfahren: Grundlagen, Auslegung, Apparate”, 3rd edition, Wiley-VCH, Weinheim 2001).

As regards separation processes specifically used for the treatment of component mixtures formed by steam cracking, in particular separation processes involving demethanization and deethanization, reference is made to the already cited article “Ethylene” in Ullmann's Encyclopedia of Industrial Chemistry. Such separation processes differ in particular in the sequence of the respective separation steps. For example, the demethanizer first process (also known as the front-end demethanizer process) and the deethanizer first process (also known as the front-end deethanizer process), as well as the depropanizer first process (also known as the front-end depropanizer process) are known. As explained in detail below, this invention is particularly suitable for use in conjunction with the deethanizer first method, but also for use with the demethanizer first method or the depropanizer first method.

In particular, demethanizers, deethanizers and depropanizers, as mentioned above, may be designed as corresponding rectification columns or may include such rectification columns, which are hereinafter also referred to as demethanization columns, deethanization columns or depropanization columns. In the language used here, “demethanizers”, “deethanizers” and “depropanizers” are understood to mean arrangements with corresponding rectification columns, to which, however, additional apparatuses, such as absorbers in deethanizers, can also be assigned. The same applies if there is talk of “demethanization”, “deethanization” or “depropanization”. If it is mentioned below that fractions “from the head” or “from the bottom” can be withdrawn from demethanizers, deethanizers and depropanizers or corresponding rectification columns, these can also be withdrawn alternatively or in addition to the rectification column from the head or from the bottom of corresponding assigned apparatus.

According to an first preferred embodiment of the present invention, the removal of hydrogen and methane in the second pre-separation step or steps also removes ethane and ethylene at least predominantly, i.e. a deethanizer-first process is carried out.

Hydrogen and methane are removed in the second pre-separation step or steps, in which ethane and ethylene are also at least predominantly removed, using a deethanization column. In this case, the fourth component mixture occurs in the area of a tray near the bottom of such a deethanization column. Compared to the second component mixture, a corresponding liquid is depleted of hydrogen, methane and hydrocarbons with two carbon atoms or corresponding components have been removed to a large extent.

As mentioned several times, as an alternative to the first preferred embodiment of the invention described above, a second advantageous embodiment is also possible in which ethane and ethylene are at least predominantly not removed during the removal of hydrogen and methane in the second pre-separation step or steps. Ethane and ethylene are therefore at least predominantly transferred to the fourth component mixture in these versions of the invention. In particular a demethanizer first method is used. If a corresponding rectification column is used to remove hydrogen and methane, the fourth component mixture is in particular a bottom liquid of such a rectification column.

The removal of hydrogen and methane in the second pre-separation step or steps, in which ethane and ethylene are at least predominantly not removed, can therefore be carried out using a demethanization column. A component mixture predominantly or exclusively containing hydrogen and methane is taken from the head of such a demethanization column or an apparatus associated with it, and a bottom liquid predominantly or exclusively containing hydrocarbons with two or more carbon atoms can be withdrawn from the bottom. This bottom liquid can in particular subsequently be deethanized, as explained below.

In other words, in a demethanizer-first process, in addition to the demethanization column, a deethanization column is foreseen. Here the third component mixture can be fed in particular into a bottom or a separation tray near the bottom of the demethanization column or into the lower area, i.e. the area of a separation tray near the bottom of the deethanization column. If a deethanizer first method is used, the third component mixture can be fed into the bottom area of the deethanization column in particular. In both cases, i.e. in a demethanizer first method and in a deethanizer first method, depropanization columns are also used. Alternatively or additionally, the third component mixture can also be fed into a depropanization column. The same applies if a depropanizer-first method is used. In this case, the third component mixture can also be fed into the depropanization column.

In particular, the hydrogen content of the first component mixture is depleted to a value of 0 to 10 mol %, in particular 0.1 to 5 mol %, for example 0.2 to 2 mol %, within the framework of the present invention in the context of the first pre-separation step or steps of the first component mixture. With such hydrogen contents, the second component mixture can also be fed to a common separation, since its other composition is sufficiently similar to a corresponding fluid from a steam cracking method. As mentioned, any remaining hydrogen can simply be removed.

As mentioned above, the first pre-separation step or steps to which the first component mixture is subjected also include an increase in pressure, in particular to an absolute pressure of 3 to 40 bar, in particular 10 to 30 bar, for example 12 to 30 bar. The pressure level depends on a pressure level at which a demethanizer or deethanizer or depropanizer is operated, as it is used in the second pre-separation step or steps, i.e. the pressure increase also carried out there takes place within the scope of the second pre-separation step or steps. Therefore, the pressure levels of the third and fourth component mixtures can be adjusted in this way in a particularly advantageous way.

Hydrogen depletion as part of the pretreatment of the first component mixture or its part subjected to the pretreatment may include in particular a partial condensation of hydrocarbons with three carbon atoms after the pressure increase or compression described above. In this way, a fraction is formed which predominantly contains hydrocarbons with three carbon atoms, but into which the other components mentioned can also partially pass. In any case, this fraction is depleted of hydrogen compared to the first component mixture. Such condensation is particularly advantageous because, as explained below, it can be carried out at least in part within the framework of this invention using refrigeration, which can be provided by process streams present in the process.

In particular, partial condensation may be carried out using refrigeration, which may be obtained at least in part by decompressing a stream predominantly containing propane. This stream, which mainly contains propane, may, for example, be the second separation product formed in the first separation step or steps. This second separation product can be expanded to produce cold and then returned to the process, in particular the propane dehydrogenation method or the steam cracking method.

It is also possible to perform partial condensation using refrigeration generated at least in part by depressurising a part of the first component mixture or its portion subject to the first pre-separation step or steps. For example, the first component mixture or its portion subjected to the first pre-separation step or steps can be fed to the first pre-separation step or steps in the form of a stream of material which is compacted and a partial stream of which is expanded downstream of compaction. The relaxed partial flow can be fed back into the compression process, so that cold can be generated continuously. So-called cold box processes, as they are generally known from the state of the art, or processes based on other separation principles can also be used in the context of this invention.

It is particularly advantageous if the propane dehydrogenation method is carried out under water-free conditions and/or in the complete absence of oxygen (also in covalently bound form and/or during regeneration). In this way it becomes possible to form the first component mixture in such a way that neither water nor oxygen-containing compounds, especially carbon dioxide, are found in it. In this way, it is particularly easy to feed a corresponding first component mixture to the first pre-separation step or steps and, in particular, to the first separation step or steps because no separation of these components is required. In other words, the first component mixture can, without separating water and carbon dioxide during the formation of the third component mixture, be fed to a rectification column, for example, which serves to deethanize and which is typically operated at cryogenic temperatures at which water and carbon dioxide would freeze out.

As mentioned above, feeding the third component mixture after the first separation step or steps to the first separation step or steps in which the third component mixture is combined with the fourth component mixture or a component mixture formed therefrom is/are particularly advantageous if the respective compositions are identical or do not differ by more than a predetermined extent.

It is therefore particularly advantageous if a hydrogen content in the third component mixture differs from a hydrogen content in the fourth component mixture by not more than 50%, in particular by not more than 25%, for example by not more than 10%, of a hydrogen content in the third component mixture and if a content of hydrocarbons having three carbon atoms, in particular propylene, in the third component mixture differs from a content of hydrocarbons having three carbon atoms, in particular propylene, in the fourth component mixture by not more than 50%, in particular by not more than 25%, for example by not more than 10%.

The present invention further extends to a plant for the production of propylene, having a first reactor unit which is provided and arranged to carry out a propane dehydrogenation method to obtain a first component mixture which contains at least hydrogen, ethane, ethylene, propane and propylene, a second reactor unit which is provided and arranged to carry out a further propylene production method, in particular a steam cracking method, to obtain a second component mixture which contains at least hydrogen, methane, ethane, ethylene, propane and propylene, and a first separation unit provided and arranged to form a separation product predominantly containing propane using at least a portion of the propane of the first and, according to a preferred execution form also the second component mixture and using one or more first separation steps, wherein means are provided arranged to supply at least a portion of the first component mixture and, according to the preferred embodiment also at least a portion of the second component mixture to the propane separation step or steps.

According to the invention, such a plant is characterised by means which are made available and set up to return the separation product, which mainly contains propane, at least in part to the further propylene production method.

With regard to the features and advantages of the inventionally proposed annex, reference is made explicitly to the features and advantages already described with regard to the explained process and its advantageous design in the explained features and advantages. The same applies in particular to an installation in accordance with a particularly preferred form of the present invention, which has means which have been set up to carry out a corresponding procedure.

The invention further comprises a process for retrofitting a plant adapted to perform a steam cracking method using a plurality of plant components such as cracking furnaces, processing equipment and separating apparatus, wherein a hydrocarbon-containing feed mixture having a first composition is fed to the plant prior to retrofitting. In accordance with the invention, the retrofitting comprises adding a hydrocarbon-containing feed mixture with a second, different composition to the plant instead of the hydrocarbon-containing feed mixture with the first composition, and using one or more of the plant components for a propane dehydrogenation method instead of for the steam cracking method, i.e. reallocating freed capacities accordingly.

An example of this is a change in the feedstock mix of the steam cracking method from heavier hydrocarbons, for example predominantly naphtha, to lighter hydrocarbons, for example ethane and/or propane and butane. While certain plant components for processing the entire product gas, such as the raw gas compressor, and plant components for processing the light product fraction, such as the demethanizer, are likely to be subjected to the same or even higher loads than before after the change in the feed mixture, other plant components, for example for processing heavier product fractions, are likely to be relieved. These relieved plant components can include the depropanizer as well as all plant components for processing a fraction of hydrocarbons with three carbon atoms including hydrogenation and a splinter. These plant components can then also be used for a propane dehydrogenation method. The procedure described in the present invention for removing hydrogen from the product gas of propane dehydrogenation is particularly advantageous here, since this procedure does not place an even greater load on the heavily utilised existing plant components of the steam cracking method, such as the demethanizer.

According to the invention, a process as described above is carried out after the retrofit and/or a corresponding plant is provided by means of the retrofit. In this way, the advantage mentioned at the beginning can be achieved that the corresponding products of a propane dehydrogenation can be purified together with the products of the steam cracking method and separate purification can be dispensed with.

The invention is explained in more detail below with reference to the attached drawings, in which a preferred form of execution of the present invention is explained compared to the state of the art.

### SHORT DESCRIPTION OF THE DRAWINGS

FIG. 1 illustrates a process designed according to an embodiment of the invention in a highly simplified, schematic representation.

In the figures, constructionally and/or functionally corresponding elements are indicated with identical reference signs and are not explained repeatedly for the sake of clarity.

### DETAILED DESCRIPTION OF THE DRAWINGS

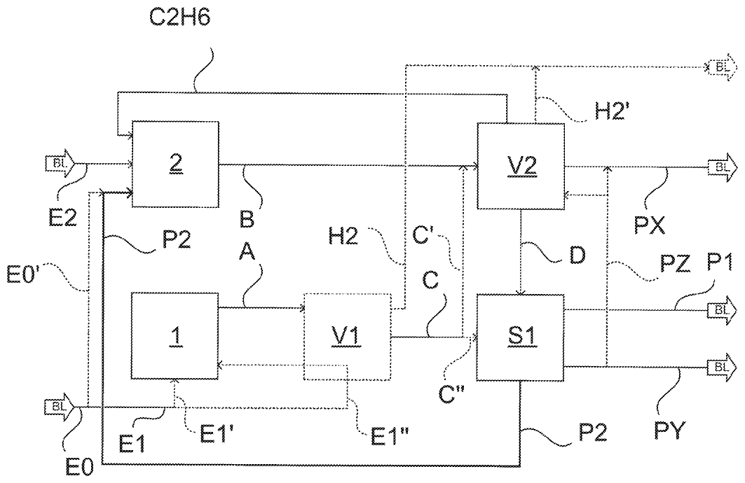

FIG. 1 illustrates a process designed according to the invention in a highly simplified, schematic representation and is designated 10 in total.

Process 10 comprises a process 1 for propane dehydrogenation, a steam cracking method 2, one or more first preliminary separation steps V1, one or more second preliminary separation steps V2, and one or more propane separation steps S1. The first pre-separation step or steps V1, the second pre-separation step or steps V2, the propane separation step or steps S1 and any other separation steps not separately illustrated here can be grouped as required and, for example, combined in corresponding plant components.

In the example shown, procedure 1 for propane dehydrogenation is supplied with an initial input stream E1, which may include propane in particular, but not recycled propane. Instead, the first input current E1 is formed exclusively using an output current E0, which is fed to process 10 from the BL system limit. A part of this output stream E0 can also be fed to the steam cracking method 2, as illustrated in the form of a dashed material stream E0′. In the example shown, the first input stream E1 is divided into two partial streams E1′ and E1″, wherein the partial stream E1′ is fed directly to process 1 for propane dehydrogenation and the partial stream E″ is first used in the first pre-separation step or steps V1. In the first pre-separation step or steps V1, the partial stream E1″ can, for example, be expanded to generate cold and only then fed into process 1 for propane dehydrogenation. In contrast to FIG. 1, however, the input stream E1 can also be treated completely as illustrated here with regard to the partial stream E1′, i.e. it can be fed directly to process 1 for propane dehydrogenation.

Process 1 for propane dehydrogenation is carried out in a generally known manner so that a first component mixture A containing at least hydrogen, ethane, ethylene, propane and propylene is formed therein and which can be carried out from process 1 for propane dehydrogenation in the form of a corresponding material stream. Procedure 1 for propane dehydrogenation may in particular be carried out using one or more suitable reactors, which may have been designed in a customary manner.

In the example shown, the first component mixture A or the corresponding material flow is at least partially fed to the first pre-separation step or steps V1 in which the first component mixture A or the corresponding material flow is subjected to a pressure increase and at least partial removal of hydrogen. As mentioned above, this can be done in a generally known way. In particular, the first component mixture A or the corresponding material flow can be liquefied in the first pre-separation step or steps V1. Separated hydrogen is illustrated in the form of a material stream called H2. Also an at least partial removal of hydrocarbons with two carbon atoms is possible, but optional, as not shown separately in FIG. 1. In this way, a component mixture is obtained, which is also referred to here as the third component mixture C, and which can be executed in the form of a corresponding material flow from the first pre-separation step or steps V1. Possible hydrogen contents of the third component mixture C or the corresponding material flow have already been explained above. In particular, the third component mixture C downstream of the first preliminary separation step or steps V1 still contains hydrocarbons with three carbon atoms and minor amounts of other components, for example hydrocarbons with two hydrocarbon atoms, if not yet removed, and hydrocarbons with four carbon atoms formed as by-products in propane dehydrogenation method 1. If the first component mixture A also contains water and carbon dioxide, these can also be removed in the first pre-separation step or steps V1.

The steam cracking method 2, which can also be carried out in the usual manner, for example by using several cracking furnaces, is fed a hydrocarbon-rich feed in the form of a material flow E2 in the example shown, which is fed from the BL plant boundary. The hydrocarbon-rich use can include in particular naphtha and lighter hydrocarbons, but also heavy hydrocarbons. In particular, hydrocarbon-rich applications may include paraffinic hydrocarbons with two, three and four carbon atoms, in particular ethane, propane and butane. The steam cracking method 2 as a whole or different furnaces used in the steam cracking method 2 can also be supplied with different hydrocarbon feedstocks and processed there under different splitting conditions. In the example shown, the steam cracking method 2 is additionally supplied with the aforementioned partial stream E0″ of the output stream E0, a propane-containing separation product P2 of the propane separation step or steps S1 and a further recirculated stream C2H6 which contains predominantly or exclusively ethane. With the exception of the return of the separation product P2, which mainly contains propane, the supply or return of the other flows shown is completely optional within the scope of this invention.

In the steam cracking method 2, the hydrocarbons of the hydrocarbon-rich feed(s) are at least partially converted so that a second component mixture B is obtained which contains at least hydrogen, methane, ethane, ethylene, propane and propylene. The second component mixture B can be drawn off from the steam cracking method 2 in the form of a corresponding material flow and then at least partly fed to one or more second pre-separation steps V2. The composition of the second component mixture B or of the corresponding material flow depends to a large extent on the hydrocarbon-rich application supplied to the steam cracking method 2.

As already explained several times, the second pre-separation step or steps V2 may include demethanization and/or deethanization in particular. For example, a demethanizer first process or a deethanizer first process can be used. Both variants have already been explained before and are generally known from the state of the art. They are therefore not explained here again. In addition to hydrogen, as illustrated here in the form of a material stream HZ, the mentioned material stream C2H6, which predominantly or exclusively contains ethane, as well as one or more product streams, here commonly designated PX, can also be formed in the second pre-separation step or steps. The product flow(s) PX can be led to the BL system limit. The hydrogen-rich material flow HZ can be combined with the hydrogen-rich material flow H2 and led together with this to the BL plant boundary. Together with the hydrogen-rich material stream HZ, methane in particular can also be discharged.

Irrespective of the process specifically carried out, however, in both cases a component mixture D is formed by the use of the second pre-separation step or steps V2, which is referred to here as the fourth component mixture, and which is at least depleted of hydrogen and methane with respect to the second component mixture B, or which is formed by at least partially removing hydrogen from methane from the second component mixture B. The second component mixture B is formed by the use of the second pre-separation step or steps V2. This fourth component mixture D is fed to the propane separation step or steps S1 in which the separation product P2, which mainly contains propane, is formed. Furthermore, in the example shown, a separation product P1 containing predominantly or exclusively propylene can also be formed in the propane separation step or steps S1 and taken to the BL plant boundary. One or more further separation products, herein referred to as PY, can also be separated in the propane separation step or steps S1 and led to the plant boundary BL. Depending on the specific design of the process in accordance with the invention, further separation steps, such as deethanization or depropanization, may be part of the second pre-separation step or steps V2 or part of the propane separation step or steps S1.

The third component mixture C can be fed to the second pre-separation step or steps V2 and/or the propane separation step or steps S1. For example, the third component mixture can be in the region of a separating bottom near the bottom of a demethanization column or deethanization column used in the second preliminary separation step or steps V2 and/or in a depropanization column which can be part of the second preliminary separation step or steps V2 and or of the propane separation step or steps S1. Corresponding alternatives are illustrated in the form of material flows C′ and C″. Material flows can also be returned from the propane separation step or steps S1 to the pre-separation step or steps V2, as illustrated here in the form of the material flow PZ.

1. A process (10) for the production of propylene, which comprises:
carrying out a propane dehydrogenation method (1) to obtain a first component mixture (A) containing at least hydrogen, ethane, ethylene, propane and propylene,
carrying out a further propylene production method (2) to obtain a second component mixture (B) containing at least hydrogen, methane, ethane, ethylene, propane and propylene,
forming a separation product (P2) predominantly containing propane using at least a portion of the propane of the first component mixture (A) and using one or more propane separation steps (S1),
wherein at least part of the first component mixture (A) is supplied to the propane separation step or steps (S1),
characterized in
that the separation product (P2) predominantly containing propane is at least partly returned to the further propylene production method (2).

2. The process (10) according to claim 1, in which the formation of the separation product (P2) predominantly containing propane which is carried out using one or more propane separation steps (S1) is further carried out using at least a part of the propane of the second component mixture (B), wherein at least a part of the second component mixture (B) is furthermore supplied to the propane separation step or steps (S1).

3. The process (10) according to claim 1 in which the predominantly containing propane separation product (P2) is not recycled to the propane dehydrogenation method (1).

4. The process (10) according to claim 1, in which only propane which has not been separated either from the first component mixture (A) or from the second component mixture (B) is supplied to the propane dehydrogenation method (1).

5. The process (10) according to claim 1, in which a steam cracking method is used as the further propylene production method (2).

6. The process (10) according to claim 1, in which the separation product predominantly containing propane predominantly contains propane and also from 0.1 to 25 volume percent mono- and polyunsaturated C3 hydrocarbons and/or heavier hydrocarbons.

7. The process (10) according to claim 1, which further includes
forming a separation product (P1) containing predominantly or exclusively propylene using at least a portion of the propylene of the first and second component mixtures (A, B) and the propane separation step or steps (S1),
forming a separation product (P3) containing at least predominantly ethylene using at least part of the ethylene of the first and second component mixtures (A, B) and one or more further separation steps; and
forming a separation product (P4) containing at least predominantly ethane using at least part of the ethane of the first and second component mixtures (A, B) and the further separation step or steps.

8. The process (10) according to claim 1, which includes
that at least part of the first component mixture (A) is subjected to one or more first pre-separation steps (V1) which comprise a pressure increase and an at least partial removal of hydrogen, to obtain a third component mixture (C),
that at least part of the second component mixture (B) is subjected to one or more second pre-separation steps (V2) which comprise a pressure increase, an at least partial removal of hydrogen and an at least partial removal of methane, to obtain a fourth component mixture (D),
that at least part of the third component mixture (C) together with at least part of the fourth component mixture (D) is subjected to the propane separation step or steps (S1), and/or
that at least part of the third component mixture (C) is subjected together with the second component mixture (B) to the second pre-separation step or steps (V2) to form the fourth component mixture (D), and the fourth component mixture (D) is subjected to the propane separation step or steps (S1).

9. The process (10) according to claim 8, in which ethane and ethylene are also at least predominantly removed during the removal of hydrogen and methane in the second preliminary separation step or steps (V2), the removal of hydrogen and methane in the second preliminary separation step or steps (V2), in which ethane and ethylene are also at least predominantly removed, being carried out using a deethanization column.

10. The process (10) according to claim 8 in which ethane and ethylene are at least predominantly not removed in the removal of hydrogen and methane in the second preliminary separation step or steps (V2), wherein the removal of hydrogen and methane in the second preliminary separation step or steps (V2) in which ethane and ethylene are at least predominantly not removed is carried out using a demethanization column.

11. The process (10) according to claim 8, in which a demethanization column and/or a deethanization column and/or a depropanization column is used in the second preliminary separation step or steps (V2) and/or in the propane separation step or steps (S1), wherein the third component mixture is fed at least partially in the liquid state into the lower region of the demethanization column and/or the deethanization column and/or the depropanization column.

12. The process (10) according to claim 8, in which in the first preliminary separation step or steps (V1) of the first component mixture its hydrogen content is depleted to a value of 0 to 10 mol %, in particular 0.1 to 5 mol %, for example 0.2 to 2 mol %.

13. The process (10) according to claim 8, in which the first pre-separation step or steps to which the first component mixture is subjected comprise an increase in pressure to an absolute pressure of 3 to 40 bar, in particular of 10 to 30 bar, for example of 12 to 30 bar, wherein in particular in the first pre-separation step or steps after the increase in pressure an at least partial condensation of components boiling lower than hydrogen is carried out.

14. A plant for the production of propylene comprising:
a first reactor unit provided and arranged to perform a propane dehydrogenation method (1) to obtain a first component mixture (A) containing at least hydrogen, ethane, ethylene, propane and propylene,
a second reactor unit which is provided and arranged to perform a further propylene production method (2) to obtain a second component mixture (B) containing at least hydrogen, methane, ethane, ethylene, propane and propylene; and
a separation unit provided and arranged to form a separation product (P2) predominantly containing propane using at least a portion of the propane of the first component mixture (A) and using one or more propane separation steps (S1),
wherein means are provided adapted to supply at least a portion of the first component mixture (A) to the propane separation step or steps (S1),
characterized by
means provided and arranged to return at least part of the separation product (P2) predominantly containing propane to the further propylene production method (2).

15. A method of retrofitting a plant arranged to perform a steam cracking method using a number of plant components, wherein a hydrocarbon-containing feed mixture having a first composition is fed to the plant prior to retrofitting, characterised in that the retrofitting comprises to feed a hydrocarbon-containing feed mixture having a second, different composition to the plant instead of the hydrocarbon-containing feed mixture having the first composition, and to use one or more of the plant components for a propane dehydrogenation method instead of for the steam cracking method, wherein after the conversion a-the process (10) according to claim 1 is used by means of the conversion.

16. The process (10) according to claim 2 in which the predominantly containing propane separation product (P2) is not recycled to the propane dehydrogenation method (1).

17. The process (10) according to claim 2, in which only propane which has not been separated either from the first component mixture (A) or from the second component mixture (B) is supplied to the propane dehydrogenation method (1).

18. The process (10) according to claim 3, in which only propane which has not been separated either from the first component mixture (A) or from the second component mixture (B) is supplied to the propane dehydrogenation method (1).

19. The process (10) according to claim 2 in which a steam cracking method is used as the further propylene production method (2).

20. A method of retrofitting a plant arranged to perform a steam cracking method using a number of plant components, wherein a hydrocarbon-containing feed mixture having a first composition is fed to the plant prior to retrofitting, characterised in that the retrofitting comprises to feed a hydrocarbon-containing feed mixture having a second, different composition to the plant instead of the hydrocarbon-containing feed mixture having the first composition, and to use one or more of the plant components for a propane dehydrogenation method instead of for the steam cracking method, wherein after the conversion the plant according to claim 14 is provided by means of the conversion.
